(19) United States
(12) Patent Application Publication
(10) Pub. No.: US 2024/0424249 A1
(43) Pub. Date: Dec. 26, 2024

(54) CATHETER ADDITIVES

(71) Applicant: ConvaTec Limited, Flintshire (GB)

(72) Inventors: Rachel Pytel, Newton, MA (US); Paul Mossman, Birkenhead (GB); Luca Barbieri, Chester (GB)

(21) Appl. No.: 18/827,101

(22) Filed: Sep. 6, 2024

Related U.S. Application Data
(63) Continuation of application No. PCT/GB2023/050545, filed on Mar. 8, 2023.
(60) Provisional application No. 63/318,013, filed on Mar. 9, 2022.

(51) Int. Cl. A61M 25/00

(52) U.S. Cl. CPC A61M 25/0017; A61M 25/0009; A61M 2205/0216; A61M 2205/0222; A61M 2205/0255; A61M 2207/10

(57) ABSTRACT

This disclosure provides an AB block copolymer amphiphilic additive comprising a hydrophobic hydrocarbon A-block and a hydrophilic poly (alkylene oxide) B-block, wherein the B-block is end-capped with a moiety that is negatively charged or that becomes negatively charged when wetted with an aqueous medium having a pH of greater than 2.

### CROSS REFERENCE TO RELATED DISCLOSURES

The present disclosure is a continuation of International Application No. PCT/GB2023/050545 filed on Mar. 9, 2023 and claims the benefit of U.S. Provisional Application No. 63/318,013 filed on Mar. 9, 2022, the disclosures of which being incorporated herein by reference in entirety.

### TECHNICAL FIELD OF THE INVENTION

The present invention relates to amphiphilic additives for intermittent catheters.

### BACKGROUND TO THE INVENTION

Intermittent urinary catheterisation is a process involving insertion of a urinary catheter through an individual's urethra and into their bladder, where it is retained to empty the bladder of urine for only the time period that is required for emptying, after which the catheter is removed. The process differs from long-term catheterisation, which makes use of an indwelling or Foley catheter that is inserted into the bladder for long periods of time (several days to months) to discharge the residual urine of the bladder continuously throughout the day.

Intermittent catheterisation is often used by patients suffering from abnormalities of the urinary system, resulting in urinary incontinence and/or a lack of control in permitting voluntary urination. Such individuals would typically make use of intermittent catheters several times a day.

Intermittent catheters are useful devices, providing users with independence and freedom to self-catheterise as and when required, without having to rely on trained personnel to be present. This, however, increases the need for intermittent catheters to be user friendly: in particular, both easy to insert and remove with minimum discomfort caused, and safe to use with features for minimising risk of infection. Users often report experiencing pain and discomfort upon insertion and/or removal of intermittent catheters. Users have, for instance, reported experiencing bladder spasms, burning sensations, and bleeding.

It is also easy for intermittent catheters to become contaminated and for bacteria to be introduced into the urethra and along the urinary tract. As a result, urinary tract infections (UTI) are common in individuals who practice intermittent catheterisation.

Surface coatings and additives for catheters have been used to help in alleviating some of these issues. US patents U.S. Pat. No. 10,058,638 B2 and U.S. Pat. No. 9,186,438 B2 describe the use of a catheter containing a polymer mixture of a base material and an amphiphilic block copolymer lubricious additive. The amphiphilic block copolymer contains both a hydrophobic and hydrophilic portion. The hydrophilic portion diffuses to the surface of the catheter due to incompatibility with the hydrophobic base material and provides for a lubricious surface coating. However, these patents do not properly address the issue of intermittent catheter bacterial contamination.

There exists a need for intermittent catheters that are both even easier and less painful to insert and remove; and that are safer to use, with features for minimising the risk of bacterial contamination and infection, especially for non-medically trained individuals practising self-catheterisation.

It is an aim of embodiments of the present invention to address one or more of the above problems by providing an intermittent catheter, suitable for self-catheterisation use, which may provide one or more of the following advantages:
A high lubricity, non-stick surface making the intermittent catheter easier to insert and remove with a lower occurrence of microtraumas in patients.
An inert, bacteria repellent surface which ensures that the catheter remains safe even when accidentally contacted by non-sterile objects, and which allows for reduced numbers of bacteria to be introduced into the urethra, thus reducing the risk of infections developing (particularly UTIs).
Not overly complex design and simple/cost-effective to manufacture.

It is also an aim of embodiments of the invention to overcome or mitigate at least one problem of the prior art, whether expressly described herein or not.

### SUMMARY OF THE INVENTION

According to a first aspect of the invention, there is provided an A-B block copolymer amphiphilic additive comprising a hydrophobic hydrocarbon A-block and a hydrophilic poly (alkylene oxide) B-block, wherein the B-block is end-capped with a moiety that is negatively charged or that becomes negatively charged when wetted with an aqueous medium having a pH of greater than 2.

In preferred embodiments, only the end-cap moiety of the additive is negatively charged or becomes negatively charged when wetted with the aqueous medium. When such an additive is used in or on an intermittent catheter, the hydrophobic block of the additive allows for hydrophobic-hydrophobic interactions between the A-block and a hydrophobic or generally hydrophobic catheter polymer, which prevents migration of the additive away from the catheter. The hydrophilic block allows for hydrophilic-hydrophilic interactions between the B-block and a hydrophilic external environment (often comprising a wetting agent aqueous medium). The hydrophilic B-block is thus able to seek towards an outer surface of the intermittent catheter due to its affinity with the hydrophilic external environment and its incompatibility with the hydrophobic catheter polymer. When the hydrophilic poly (alkylene oxide) B-block is present at or on the outer surface of the intermittent catheter, it enables wetting of the outer surface simply by applying water or gel to create a lubricious coating. Further, the poly (alkylene oxide) B-block is not harmful to the user and poses minimal risk to the user of developing sensitisation.

The end-cap moiety may be negatively charged or able to become negatively charged when wetted with an aqueous medium of pH >2, and may provide at least one of the following advantages:
The outer layer of the urothelium contains a glycosaminoglycan (GAG) layer that is believed to be negatively charged. The negative charge on the end-cap of the additive causes a repulsive interaction between the additive and the negatively charged GAG layer of the urothelium when the catheter is inserted, in use. This results in a low detachment force between the additive and GAG layer, which allows the catheter to be inserted and removed smoothly and with minimal “sticking” of the additive to the urothelium, thus leading to minimal occurrence of urethral microtraumas.
Where the catheter is wetted prior to use with an aqueous medium (e.g. wetting agent), the negative charge on the end-cap is able to form strong charged hydrogen bonds with water molecules in the aqueous medium (3-5 times stronger than neutral hydrogen bonds). This significantly reduces the speed at which the catheter dries, which keeps the catheter lubricated for longer and further decreases “sticking” effects between the catheter and the urothelium.
The negative charge on the end-cap of the additive is also able to repel bacterial membranes which are typically negatively charged due to the presence of highly electronegative groups on their constituent phospholipids and lipopolysaccharides. The negatively charged end-cap helps minimise bacterial attachment to the catheter, greatly decreasing the risk of the user developing UTIs.

In some embodiments, the additive comprises a poly (alkylene oxide) B-block formed from alkylene oxide monomer units that are independently selected from the group consisting of: ethylene oxide, propylene oxide, and combinations thereof. In preferred embodiments, all of the monomer units are ethylene oxide or all of the monomer units are propylene oxide. Most preferably, the poly (alkylene oxide) of the B-block is polyethylene oxide.

Such poly (alkylene oxides) are non-toxic and pose minimal harm/risk of sensitisation to users of catheters comprising the additive.

In some embodiments, the poly (alkylene oxide) B-block comprises at least 1, 2, 3, 4, or at least 5 monomer units. In some embodiments, the poly (alkylene oxide) B-block comprises no greater than 20, 19, 18, 17, 16, 15, 14, 13, 12, 11, 10, 9, 8, 7, or no greater than 6 monomer units. In some embodiments, the poly (alkylene oxide) B-block comprises between 1 and 15 monomer units, preferably between 1 and 10 monomer units, or between 2 and 10 monomer units.

In some embodiments, the end-cap moiety may be inherently negatively charged. The end-cap moiety may comprise a stabilised anion. The anion may be stabilised by one or more of the group consisting of: hyperconjugative/inductive effects, resonance/aromaticity effects, steric bulk, electronegative atoms, and combinations thereof.

In other embodiments, the end-cap moiety may become negatively charged when wetted with an aqueous medium having a pH of greater than 2, 2.5, or preferably of greater than 3, 3.5, 4, 4.5, 5, 5.5, 6, 6.5, 7, 7.5, 8, 8.5 or of greater than 9. In some embodiments, the aqueous medium may have a pH of no greater than 13, or of no greater than 12.5, 12, 11.5, 11, 10.5, or of no greater than 10. The aqueous medium may have a pH of between 2-13, 2-12, 2-11, 2-10, 2-9, 2-8, 2-7, or of between 3-13, 3-12, 3-11, 3-10, 3-9, 3-8, 3-7, or of between 4-13, 4-12, 4-11, 4-10, 4-9, 4-8, 4-7, or of between 5-13, 5-12, 5-11, 5-10, 5-9, 5-8, 5-7, or of between 6-13, 6-12, 6-11, 6-10, 6-9, 6-8, or of between 6-7. The B-block may be end-capped with an ionisable moiety that becomes negatively charged when wetted with the aqueous medium. The ionisable moiety may preferably be a deprotonatable moiety (preferably an acid) that deprotonates and becomes negatively charged when wetted with the aqueous medium. In some embodiments, the end-cap moiety has a pKa of at least −10, or at least −8, −6, −4, −2, 0, 2, or at least 4. The end-cap moiety may have a pKa of no greater than 12, or preferably of no greater than 10, or of no greater than 8, or more preferably of no greater than 7, 6, 5, 4, 3, 2, or of no greater than 1. The end-cap moiety may have a pKa of between −10 and 12, or between −5 and 10, or between 0 and 8, or 0 and 7, or preferably between 1 and 7, or 1 and 6, or 2 and 7, or 2 and 6, or 1 and 5, or 2 and 5, or between 3 and 5.

The end-cap moiety may preferably comprise at least one electronegative atom, which may be independently selected from the group consisting of: oxygen, sulfur, nitrogen, a halogen, and combinations thereof. In some embodiments, the end-cap moiety is independently selected from the group consisting of: a carboxylic acid, a carboxylate, a sulfonic acid, a sulfonate, a phosphonic acid, a phosphonate, a stabilised phenolic acid, a stabilised phenolate, and salts or derivatives thereof. Preferably, the B-block is end-capped with a carboxylate-containing moiety.

The hydrophobic A-block may comprise a carbon chain of at least 5 carbon atoms, or at least 10, 15, 20, 25, 30, 35, or 40 carbon atoms. The hydrophobic portion may preferably comprise a carbon chain of between 20-52 carbon atoms.

In some embodiments, the A-block comprises a hydrocarbon chain block of the formula CH₃CH₂(CH₂CH₂)ₐ. The value of “a” may be between 5-25; for instance, “a” may be 5, 6, 7, 8, 9, 10, 11, 12, 13, 14, 15, 16, 17, 18, 19, 20, 21, 22, 23, 24, or 25, or a half integer of any of the above values. The value of “a” may preferably be between 9-25; for instance, “a” may be 9, 10, 11, 12, 13, 14, 15, 16, 17, 18, 19, 20, 21, 22, 23, 24, or 25, or a half integer of any of the above values.

In some embodiments, one or both of the hydrophobic hydrocarbon A-block and the hydrophilic B-block may be branched. The hydrophobic A-block may comprise hydrophobic hydrocarbon chains branching therefrom. The hydrophobic hydrocarbon chains may be of shorter chain lengths than the hydrophobic hydrocarbon A-block. The hydrophilic B-block may comprise further hydrophilic B-blocks branching therefrom. According to a second aspect of the invention, there is provided an intermittent catheter comprising a hollow polymeric tubular body comprising a base polymer and a copolymer amphiphilic additive comprising at least one hydrophobic portion and at least one hydrophilic portion, wherein at least one hydrophilic portion comprises a moiety at a terminal end thereof that is negatively charged or that becomes negatively charged when wetted with an aqueous medium having a pH of greater than 2.

The additive may be a block copolymer comprising at least one hydrophobic portion in the form of at least one hydrophobic A-block and further comprising at least one hydrophilic portion in the form of at least one hydrophilic B-block, wherein at least one hydrophilic B-block comprises a moiety at a terminal end thereof that is negatively charged or that becomes negatively charged when wetted with an aqueous medium having a pH of greater than 2.

In some embodiments, the additive is graft copolymer. The graft copolymer may comprise a hydrophobic portion (which may be a hydrophobic A-block) with hydrophilic portions (which may be hydrophilic B-blocks) branching therefrom, wherein at least one hydrophilic portion comprises a moiety at its terminal end that is negatively charged or that becomes negatively charged when wetted with the aqueous medium. Alternatively, the graft copolymer may comprise a hydrophilic portion with hydrophobic portions branching therefrom.

In further embodiments, the additive is a brush copolymer. The additive may comprise a single hydrophilic portion (which may be a B-block) with more than one hydrophobic portion (which may be A-blocks) branching from an end thereof. In the respective embodiments, the B-block or A-block may comprise 2, 3, 4, 5, 6, 7, 8, 9, 10, or more hydrophobic A-blocks or hydrophilic B-blocks branching from the end thereof. In all embodiments, at least one hydrophilic portion comprises a moiety at its terminal end that is negatively charged or that becomes negatively charged when wetted with the aqueous medium.

In further embodiments, the additive is a star-block or a multi-block copolymer comprising hydrophilic and hydrophobic monomer units.

In some embodiments, the additive is a B-A-B tri-block copolymer comprising a hydrophobic hydrocarbon A-block and hydrophilic B-blocks, wherein at least one hydrophilic B-block comprises a moiety at its terminal end that is negatively charged or that becomes negatively charged when wetted with the aqueous medium.

In preferred embodiments, the additive is an A-B block copolymer comprising a hydrophobic A-block and a hydrophilic B-block, wherein the hydrophilic B-block comprises a moiety at its terminal end that is negatively charged or that becomes negatively charged when wetted with an aqueous medium having a pH of greater than 2.

Statements of invention below relating to the additive or a part thereof may be applied mutatis mutandis to each of the copolymer forms above.

The amphiphilic additive of the second aspect of the invention is preferably the additive of the first aspect of the invention. Statements of invention above relating to first aspect of the invention may equally be applied to the second aspect of the invention.

The hydrophobic portion of the additive may be or comprise a hydrophobic A-block, preferably as described for the first aspect of the invention.

In preferred embodiments, the amphiphilic additive is only negatively charged or only becomes negatively charged when wetted with the aqueous medium, at the terminal end of the or of at least one B-block.

The number of monomer units in the or at least one B-block of the additive may preferably be as described for the first aspect of the invention above. At least one monomer unit in the or at least one B-block may be independently selected from the group consisting of: alkylene oxides, alkylene glycols, epihalohydrins, alkylene imines, lactones, vinyl alcohol, and vinyl alkanoates. At least one monomer unit in the or at least one B-block may be independently selected from the group consisting of: ethylene oxide, propylene oxide, ethylene glycol, propylene glycol, epichlorohydrin, ethylene imine, caprolactone, vinyl alcohol, and vinyl acetate. In some embodiments, the additive comprises a poly (alkylene oxide) B-block formed from alkylene oxide monomer units that are independently selected from the group consisting of: ethylene oxide, propylene oxide, and combinations thereof. In preferred embodiments, all of the monomer units are ethylene oxide or all of the monomer units are propylene oxide. Most preferably, the poly (alkylene oxide) of the B-block is polyethylene oxide.

Statements of invention relating to the end-cap moiety of the first aspect of the invention may also be applied mutatis mutandis to the moiety that is negatively charged or that becomes negatively charged when wetted in the additive of the second aspect of the invention.

In some embodiments, less than or equal to 75% of the total number of B-block monomer units are negatively charged or become negatively charged when wetted with the aqueous medium, or less than or equal to 70, 65, 60, 55, or preferably less than or equal to 50, 45, 40, 35, 30, 25, 20, 15, 10, 9, 8, or less than or equal to 7% of the total number of B-block monomer units are negatively charged or become negatively charged when wetted with the aqueous medium. In such embodiments, the monomer units that are negatively charged or that become negatively charged when wetted with the aqueous medium are preferably at the terminal end of the B-block. In some embodiments, the or at least one B-block comprises between 2 and 15 monomer units, preferably between 2 and 10 monomer units; and the total number of B-block monomer units that are negatively charged or that become negatively charged are as described by the percentages above.

In preferred embodiments, the or at least one B-block is end-capped with a moiety that is negatively charged or that becomes negatively charged when wetted with the aqueous medium. The end-cap moiety may preferably be as described for the first aspect of the invention above. In preferred embodiments, only the end-cap moiety of the additive is negatively charged or becomes negatively charged when wetted with the aqueous medium.

In some embodiments, the moiety may become negatively charged when wetted with an aqueous medium having a pH of greater than 2, 2.5, or preferably of greater than 3, 3.5, 4, 4.5, 5, 5.5, 6, 6.5, 7, 7.5, 8, 8.5, or of greater than 9. In some embodiments, the aqueous medium may have a pH of no greater than 13, or of no greater than 12.5, 12, 11.5, 11, 10.5, or of no greater than 10. The aqueous medium may have a pH of between 2-13, 2-12, 2-11, 2-10, 2-9, 2-8, 2-7, or of between 3-13, 3-12, 3-11, 3-10, 3-9, 3-8, 3-7, or of between 4-13, 4-12, 4-11, 4-10, 4-9, 4-8, 4-7, or of between 5-13, 5-12, 5-11, 5-10, 5-9, 5-8, 5-7, or of between 6-13, 6-12, 6-11, 6-10, 6-9, 6-8, or of between 6-7.

The aqueous medium may have a pH that is greater than or equal to the pKa of the moiety. The aqueous medium may have a pH of at least 0.5 pH units above the pKa of the moiety, or at least 1 pH unit above the pKa, or at least 1.5, or preferably at least 2 pH units above the pKa, or at least 2.5, 3, 3.5, or at least 4 pH units above the pKa of the moiety. The aqueous medium may have a pH that is less than 12 pH units above the pKa of the moiety, or less than 11, 10, 9, 8, 7, 6, or less than 5 pH units above the pKa of the moiety.

In some embodiments, the aqueous medium is a liquid. The aqueous medium may be an aqueous solution or water and may optionally comprise one or more ingredients independently selected from the group consisting of: a salt, a buffer, an antibiotic, an active agent (which may be a medicament), a thickening agent, a volatile agent, and combinations thereof.

In some embodiments, the aqueous medium is a gel. The aqueous medium may be a colloid comprising particles dispersed in a liquid medium.

In some embodiments, the aqueous medium may have a viscosity of greater than 0.5 cP, or of greater than 1, 1.5, 2, 3, 4, 5, 10, 20, 30, 40, 50, 75, 100, 150, 200, 300, 400, 500, 600, 700, 800, 900, or of greater than 1000 cP. The aqueous medium may have a viscosity of no greater than 100000 cP, or of no greater than 90000, 80000, 70000, 60000, 50000, 40000, 30000, 20000, 10000, 5000, 4000, 3000, 2000, 1000, 500, 400, 300, 200, 100, 50, 25, 10, 5, 4, 3, 2, or of no greater than 1 cP. The aqueous medium may have a viscosity of between 0.5-5000 cP, or of between 0.5-2500, 0.5-1000, 1-1000, 10-1000, 50-1000, 100-1000, 500-1000, 0.5-500, 1-500, 10-500, 50-500, 100-500, 250-500, 0.5-250, 1-250, 10-250, 50-250, or of between 100-250 cP.

The aqueous medium may be a catheter wetting agent. The aqueous medium may both cause the moiety on the additive to become negatively charged and encourage the hydrophilic B-blocks of the additives to seek towards an outer surface of the intermittent catheter due to their affinity with the hydrophilic aqueous medium, which further enhances the lubricating effect of the additive.

The aqueous medium may be applied, in use, to a surface of the intermittent catheter, preferably an outer surface. In some embodiments, the intermittent catheter is submerged in the aqueous medium.

In preferred embodiments, the intermittent catheter base polymer is hydrophobic or partly hydrophobic. A hydrophobic base polymer facilitates increased hydrophobic-hydrophobic interactions between the hydrophobic portion of the additive and the base polymer. This further decreases the energetic favourability for the hydrophobic portion to leave the base polymer and migrate out into the more hydrophilic external environment.

In some embodiments, the base polymer is formed from at least one material that is independently selected from the group consisting of: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, styrene-butadiene copolymer (SBC), styrene-ethylene-butylene-styrene copolymer (SEBS), water disintegrable or enzymatically hydrolysable material, and combinations, blends or copolymers of any of the above materials.

In preferred embodiments, the base polymer is formed from at least one material that is independently selected from the group consisting of: polyolefins, polyesters, polyacrylates, polyamides, thermoplastic elastomeric material, polyether block amide, thermoplastic vulcanizates, thermoplastic copolyesters, thermoplastic polyamides, fluororubber, water disintegrable or enzymatically hydrolysable material, and combinations, blends or copolymers of any of the above materials.

In some embodiments, said water disintegrable or enzymatically hydrolysable material may be independently selected from the group consisting of: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly (methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), and combinations, blends or co-polymers of any of the above materials.

In some preferred embodiments, the base polymer is formed from at least one polyolefin material, preferably comprising polyethylene and/or polypropylene.

In some preferred embodiments, the base polymer is formed from at least one thermoplastic elastomeric material. The base polymer may preferably comprise a thermoplastic polyolefin.

In some embodiments, the additive is homogeneously distributed with the polymer. The additive may be uniformly distributed throughout the base polymer.

At least some of the additive may be at or on a surface of the body, preferably an outer surface of the body. By “at the outer surface”, it is meant that at least a portion of the additive forms part of the surface or protrudes from the surface. In some embodiments, part of the additive is retained or anchored in the body while part of the additive forms part of or protrudes from the outer surface of the body.

The outer surface may comprise at least one of the group consisting of: the external facing surface of the body, the lumen of the body, and any eyelets present on the body. In preferred embodiments, the outer surface is the external-facing surface of the body and/or the inner lumen. In some embodiments, the outer surface may comprise the external-facing surface of the body of the catheter, the inner lumen, and the eyelets.

The additive may be concentrated at or on the outer surface of the body. For example, at least 25%, 30%, 35%, 40%, 45%, 50%, 55%, 60%, 65%, 70%, 75%, 80%, 85%, 90%, or at least 95% of the number of additive copolymers may be at or on the outer surface of the body.

In some embodiments, at least 25%, 30%, 35%, 40%, 45%, 50%, 55%, 60%, 65%, 70%, 75%, 80%, 85%, 90%, or at least 95% of the number of additive copolymers may have hydrophilic portions that are at or on the outer surface of the body.

In some embodiments, the additive is located at and/or on at least 50, 55, 60, 65, 70, 75, 80, 85, 90, 95, 96, 97, 98 or at least 99% of the outer surface area of the polymeric tubular body, preferably at least 75% or at least 90% of the outer surface area of the polymeric tubular body or between 75% and 100% of the outer surface area.

In some embodiments, the moieties that are negatively charged or that become negatively charged when wetted with the aqueous medium are present at and/or on at least 5% of the outer surface area of the body, or at least 10, 15, 20, 25, 30, 35, 40, 45, 50, 55, 60, 65, 70, 75, 80, 85, 90, 95, 96, 97, 98, or at least 99% of the outer surface area of the body.

In some embodiments, the additive is present at a concentration of at least 0.1, 0.2, 0.3. 0.4. 0.5, 0.75, 1, 2, 3, 4, 5, 10, 15 or at least 20% by weight of the combination of base polymer and additive. The additive may be present at a concentration of between 0.1-20%, or between 0.5-15% or 0.5-5% by weight of the combination of base polymer and additive.

In some embodiments, the additive comprises a layer that is on or that comprises a surface of the body, preferably the outer surface.

The layer comprising the additive may be on the surface of the body. In some embodiments, the layer comprising the additive is substantially separate from the body and the layer may be bonded to the body or may be coating the body. The layer may be bonded to the body via covalent bonds, ionic bonds, hydrogen bonds, or Van der Waals forces. The additive may be bonded to the body via one or more surface linker groups which may be present on the additive, the body of the intermittent catheter or both. In some embodiments, the layer comprising the additive may comprise the surface of the body. In such embodiments the layer may form the surface of the body. The layer may comprise a co-extruded layer which is melded with or is physically entangled with the body, and this may form an integral layer. The layer of additive may be integrally formed with the body.

In some embodiments, polymer diffusion occurs between the layer comprising the additive and the catheter body. The layer and the body may be held together by polymer chains extending across the interface between the layer and body. In some embodiments, the additive infiltrates the catheter body.

In some embodiments, the layer comprising the additive comprises or is on an inner surface of the body, an outer surface of the body, or both. The inner surface of the body may comprise a lumen of the intermittent catheter. In preferred embodiments, the layer comprising the additive comprises or is on at least an outer surface of the body.

In some embodiments, the layer comprising the additive is on or comprises at least 50, 55, 60, 65, 70, 75, 80, 85, 90, 95, 96, 97, 98 or at least 99% of the or each surface area of the body, preferably at least 75% or at least 90% of the or each surface area or between 75% and 100% of the or each surface area. In embodiments in which the layer comprising the additive comprises or is on both an inner and outer surface of the body, the additive may comprise at least 50, 55, 60, 65, 70, 75, 80, 85, 90, 95, 96, 97, 98 or at least 99% of each surface area of the body, preferably at least 75% or at least 90% of each surface area or between 75% and 100% of each surface area of both surfaces.

In some embodiments, at least 75% of the layer comprising the additive, or at least 76, 77, 78, 79, 80, 81, 82, 83, 84, 85, 86, 87, 88, 89, 90, 91, 92, 93, 94, 95, 96, 97, 98, 99, or 100% of the layer is the additive.

In some embodiments, the layer comprising the additive has an additive concentration of at least 0.1, 0.2, 0.3. 0.4. 0.5, 0.75, 1, 2, 3, 4, 5, 10, 15 or at least 20% by weight of the combination of base polymer and additive.

In some embodiments, the layer comprising the additive has an additive concentration of no greater than 70, 65, 60, 65, 60, 55, or of no greater than 50% by weight of the combination of the base polymer and additive.

The layer comprising the additive may have an additive concentration of greater than 5% by weight of the combination of base polymer and additive. The layer may have an additive concentration of between 6-50% by weight of the combination of base polymer and additive.

The layer comprising the additive may have an additive concentration of between 10-50% by weight of the combination of base polymer and additive, or of between 15-50, 20-50, 25-50, 30-50, 35-50, 40-50, or of between 45-50% by weight of the combination of base polymer and additive.

The layer comprising the additive may have an additive concentration of between 6-45% by weight of the combination of base polymer and additive, or of between 6-40, 6-35, 6-30, 6-25, 6-20, 6-15, or of between 6-10% by weight of the combination of base polymer and additive.

The layer comprising the additive may have an additive concentration of between 10-45% by weight of the combination of base polymer and additive, or of between 15-45, 20-45, 25-45, 30-45, 35-45, 40-45, 10-40, 15-40, 20-40, 25-40, 30-40, 35-40, 10-35, 15-35, 20-35, 25-35, 30-35, 10-30, 15-30, 20-30, 25-30, 10-25, 15-25, 20-25, 10-20, 15-20, or of between 10-15% by weight of the combination of base polymer and additive.

In some embodiments, the layer comprising the additive has a thickness of at least 1 μm, or of at least 2, 3, 4, 5, 6, 7, 8, 9, 10, 15, 20, 25, 30, 35, 40, 45, or of at least 50 μm. In some embodiments, the layer comprising the additive has a thickness of no more than 10000 μm, or of no more than 9000, 8000, 7000, 6000, 5000, 4000, 3000, 2000, 1000, 900, 800, 700, 600, 500, 400, or of no more than 300 μm.

In some embodiments, the layer comprising the additive has a thickness of between 50-300 μm.

The layer comprising the additive may have a thickness of between 60-300 μm, or of between 80-300, 100-300, 120-300, 140-300, 160-300, 180-300, 200-300, 220-300, 240-300, 260-300, or of between 280-300 μm.

The layer comprising the additive may have a thickness of between 50-280 μm, or of between 50-260, 50-240, 50-220, 50-200, 50-180, 50-160, 50-140, 50-120, 50-100, 50-80, or of between 50-60 μm.

The layer comprising the additive may have a thickness of between 60-280 μm, or of between 80-280, 100-280, 120-280, 140-280, 160-280, 180-280, 200-280, 220-280, 240-280, 260-280, 60-260, 80-260, 100-260, 120-260, 140-260, 160-260, 180-260, 200-260, 220-260, 240-260, 60-240, 80-240, 100-240, 120-240, 140-240, 160-240, 180-240, 200-240, 220-240, 60-220, 80-220, 100-220, 120-220, 140-220, 160-220, 180-220, 200-220, 60-200, 80-200, 100-200, 120-200, 140-200, 160-200, 180-200, 60-180, 80-180, 100-180, 120-180, 140-180, 160-180, 60-160, 80-160, 100-160, 120-160, 140-160, 60-140, 80-140, 100-140, 120-140, 60-120, 80-120, 100-120, 60-100, 80-100, or of between 60-80 μm.

In some embodiments, an outer surface of the polymeric tubular body comprises a separate or further lubricating agent or bacteria-repellent agent at and/or on the surface, in addition to the additive. The separate or further lubricating agent or bacteria-repellent agent may be bonded at and/or on the surface.

In some embodiments, said further lubricating agent or bacteria-repellent agent is formed from a coating material selected from the group consisting of: silver-based, polytetrafluoroethylene, hydrogel, silicone, lecithin, salicylic acid, minocycline, rifampin, fluorinated ethylene propylene, polyvinylidone, polyvinyl compounds, polylactames, polyvinyl pyrrolidones, polysaccharides, heparin, dextran, xanthan gum, derivatised polysaccharides, hydroxy propyl cellulose, methyl cellulose, polyurethanes, polyacrylates, polyhydroxyacrylates, polymethacrylates, polyacrylamides, polyalkylene oxides, polyethylene oxides, polyvinyl alcohols, polyamides, polyacrylic acid, hydroxy ethylmethyl acrylate, polymethylvinyl ether, maleinic acid anyhydride, penicillin, neomycin sulfate, cephalothin, Bacitracin, phenoxymethyl penicillin, lincoymycin hydrochloride, sulfadiazine, methyl sulfadiazine, succinoylsulfathiazole, phthalylsulfathiazde, sulfacetamine, procaine penicillin, streptomycin, aureomycin, terramycin, terramycin, quaternary ammonium halides, cetyl pyridinium chloride, triethyl dodecyl ammonium bromide, hexachlorophene and nitrofurazone, or any combination thereof.

In some embodiments, the catheter comprises at least one further amphiphilic additive. The further amphiphilic additive may be an A-B block copolymer but without a moiety that is negatively charged or that becomes negatively charged when wetted with the aqueous medium. Statements of invention above relating to the additives of the invention may also be applied to at least one further additive, bar statements relating to negatively charged moieties or moieties that become negatively charged when wetted with the aqueous medium. In some embodiments, at least one further additive may be identical to the inventive additive except for it not containing a moiety that is negatively charged or that becomes negatively charged when wetted with the aqueous medium.

In some embodiments, the additives of the invention comprising a moiety that is negatively charged or that becomes negatively charged when wetted with the aqueous medium comprise at least 0.5% of the total number of additives, or at least 1, 2, 3, 4, 5, 6, 7, 8, 9, 10, 15, 20, 25, 30, 35, 40, 45, 50, 55, 60, 65, 70, 75, 80, 85, 90, 95, 96, 97, 98 or at least 99%, or substantially 100% of the total number of additives. The additives of the invention may comprise no greater than 95, 90, 85, 80, 75, 70, 65, 60, 55, 50, 45, 40, 35, 30, 25, 20, 15, 10, 9, 8, 7, 6, or no greater than 5% of the total number of additives.

According to a third aspect of the invention, there is provided a method of manufacturing an intermittent catheter, the method comprising the steps of:
(a) Providing a base polymer and a copolymer amphiphilic additive comprising at least one hydrophobic portion and at least one hydrophilic portion, wherein at least one hydrophilic portion comprises a moiety at a terminal end thereof that is negatively charged or that becomes negatively charged when wetted with an aqueous medium having a pH of greater than 2; and
(b) Forming a hollow polymeric tubular catheter body comprising the base polymer and the additive.

The intermittent catheter may preferably be the intermittent catheter of the second aspect of the invention. Statements of invention relating to the intermittent catheter of the second aspect of the invention may also be applied to the third aspect of the invention.

The amphiphilic additive may preferably be the amphiphilic additive of the first and/or second aspects of the invention. Statements of invention relating to the additives of the first and second aspects of the invention may also be applied to the third aspect of the invention. In preferred embodiments, the additive is an A-B block copolymer comprising a hydrophobic A-block and a hydrophilic B-block, wherein the hydrophilic B-block comprises a moiety at its terminal end that is negatively charged or that becomes negatively charged when wetted with an aqueous medium having a pH of greater than 2.

The aqueous medium is preferably the aqueous medium of the first and second aspects of the invention. Statements of invention relating to the aqueous medium of the first and second aspects of the invention may also be applied to the third aspect of the invention.

Formation of the hollow polymeric tubular body may comprise a melt-extrusion or injection-moulding procedure. In some embodiments, the method may comprise mixing the base polymer and additive together to form a mixture, and melt-extruding or injection-moulding the mixture to form the catheter body.

The base polymer and/or additive, preferably both, may be provided in granulate or powder form. The method may comprise mixing the granulate or powder base polymer and additive to form a mixture, and melt-extruding or injection-moulding the mixture to form the hollow polymeric tubular intermittent catheter body.

In some embodiments, the method comprises melting the mixture of the base polymer and additive to form a second mixture before melt-extruding or injection-moulding the second mixture to form the hollow polymeric tubular intermittent catheter body.

The method may comprise extruding the base polymer and the additive to form a hollow polymeric tubular body comprising the base polymer and a layer comprising the additive on or comprising a surface of the catheter body, preferably an outer surface.

In some embodiments, the method comprises co-extruding the base polymer and the layer comprising the additive simultaneously.

In some embodiments, the method comprises co-extruding the layer comprising the additive using direct, indirect, hydrostatic, or impact co-extrusion.

In some embodiments, the method comprises co-extruding the layer comprising the additive using cold, warm, hot, or friction co-extrusion.

In other embodiments, the method comprises extrusion coating the additive on the surface of the catheter body. The surface preferably comprises an outer surface of the body.

The base polymer and/or additive, preferably both, may be provided in granulate or powder form prior to extrusion.

The method may comprise melt-extruding the base polymer to form the hollow polymeric tubular body, and separately melt-extruding the additive onto a surface (preferably the outer surface) of the hollow polymeric tubular body.

The method may comprise using a blown or cast film process to coat the layer comprising an additive as a molten web of synthetic resin onto the surface of the intermittent catheter body after its formation.

The method may comprise extruding a molten layer comprising an additive from a slot die directly onto a tacky catheter body. The tacky catheter body may be moved beneath the die on extrusion coating of the additive to form the layer comprising an additive on the outer surface of the catheter.

Alternatively, the method may comprise co-extruding (melt extruding) both the base polymer and additive substantially simultaneously, so that a layer comprising the additive is formed as a layer on the base polymer, or comprises the surface of the base polymer.

The method may comprise melting both a base polymer mixture and additive and delivering a steady volumetric throughput of both the mixture and additive to a single extrusion head under pressure, which allows for co-extrusion of the layer comprising an additive and the base polymer simultaneously. The co-extrusion may employ elevated temperature and pressure causing entanglements to form between the base polymer chains and additive molecules.

The method may comprise mixing the granulate or powder base polymer with a further additive, as described in the second aspect of the invention, to form a mixture, and melt-extruding the mixture to form the hollow polymeric tubular body.

In some embodiments, the method comprises melting the mixture of the base polymer and further additive to form a second mixture before extruding the second mixture to form the hollow polymeric tubular intermittent catheter body.

According to a fourth aspect of the invention, there is provided the use of a copolymer amphiphilic additive comprising at least one hydrophobic portion and at least one hydrophilic portion, wherein at least one hydrophilic portion comprises a moiety at a terminal end thereof that is negatively charged or that becomes negatively charged when wetted with an aqueous medium having a pH of greater than 2, as a bacteria-repellent and/or lubricant in or on an intermittent catheter.

The amphiphilic additive may preferably be the amphiphilic additive of the first and/or second aspects of the invention. Statements of invention relating to the additives of the first and second aspects of the invention may also be applied to the fourth aspect of the invention. In preferred embodiments, the additive is an A-B block copolymer comprising a hydrophobic A-block and a hydrophilic B-block, wherein the hydrophilic B-block comprises a moiety at its terminal end that is negatively charged or that becomes negatively charged when wetted with an aqueous medium having a pH of greater than 2.

The aqueous medium is preferably the aqueous medium of the first and second aspects of the invention. Statements of invention relating to the aqueous medium of the first and second aspects of the invention may also be applied to the fourth aspect of the invention.

The intermittent catheter may preferably be the intermittent catheter of the second aspect of the invention. Statements of invention relating to the intermittent catheter of the second aspect of the invention may also be applied to the fourth aspect of the invention. The intermittent catheter may be manufactured according to the method of the third aspect of the invention.

According to a fifth aspect of the invention, there is provided a packaged intermittent catheter comprising a packaging container in which is located an intermittent catheter of the second aspect of the invention, and optionally a wetting agent.

Statements of invention above relating to the intermittent catheter of the second aspect of the invention or to any of its components may also be applied to the fifth aspect of the invention.

The wetting agent, when present, may surround the intermittent catheter or may be separated from the intermittent catheter within the packaging, for example by providing the wetting agent in a separate container within the packaging container.

The wetting agent may comprise the aqueous medium of the previous aspects of the invention. Statements of invention above relating to the aqueous medium of the previous aspects of the invention may also be applied to the fifth aspect of the invention.

In some embodiments, the aqueous medium is contained within a separate container, such as a bag or sachet, within the container housing and is not in direct contact with the intermittent catheter.

The separate container may be pierceable, in use, to release the contained aqueous medium from the separate container and into direct contact with the intermittent catheter.

Prior to opening the packaging container, prior to removing the intermittent catheter and/or prior to inserting the intermittent catheter, the user may release the aqueous medium from the separate container and apply the medium to the outer surface of the catheter. The solution may assist in both causing the amphiphilic additive to become negatively charged and in increasing the lubricity of the catheter surface.

### DETAILED DESCRIPTION OF THE INVENTION

In order that the invention may be more clearly understood an embodiments thereof will now be described, by way of example only:

### Example 1

A first embodiment of an intermittent catheter of the invention is provided by an intermittent catheter containing a hollow polymeric tubular body comprising a base polymer formed of thermoplastic polypropylene and further comprising an A-B block copolymer amphiphilic additive of the formula

CH₃CH₂(CH₂CH₂)₂₀(OCH₂CH₂)₈COOH.

The additive comprises a polyethylene oxide hydrophilic portion which seeks towards the outer surface of the body due to its incompatibility with the hydrophobic base polymer, the outer surface becoming lubricious as a result. The additive also comprises a polyethylene lipophilic and hydrophobic portion which ensures that the hydrophilic portion is secured to the base polymer.

The intermittent catheter may be prepared as described in US patents U.S. Pat. No. 10,058,638 B2 and U.S. Pat. No. 9,186,438 B2.

Before use, an aqueous medium with a pH of greater than 2 (preferably greater than 3.5), which may also function as a wetting agent, is contacted with the outer surface of the intermittent catheter. This may be performed by the user shortly before use or the intermittent catheter may alternatively be packaged submerged in the aqueous medium and ready for use.

The aqueous medium deprotonates the end-cap carboxylic acid moiety to generate a negatively charged carboxylate.

The end-cap carboxylate moiety provides at least the following advantages:
The negative charge on the end-cap of the additive repels the negatively charged GAG layer of the urothelium when the catheter is inserted. This results in a low detachment force between the additive and GAG layer, which allows the catheter to be inserted and removed smoothly and with minimal “sticking” of the additive to the urothelium, thus leading to minimal occurrence of urethral microtraumas.
The negative charge on the end-cap is also able to form strong charged hydrogen bonds with water molecules in the aqueous medium. This reduces the speed at which the catheter dries and keeps the catheter lubricated for longer, further decreasing “sticking” effects between the catheter and the urothelium.
The negative charge on the end-cap of the additive is also able to repel bacterial membranes, which helps minimise bacterial attachment to the catheter, greatly decreasing the risk of the user developing UTIs.

The intermittent catheter of Example 1 was less susceptible to bacterial attachment compared with a catheter comprising a similar additive which had a terminal —OH group instead of the carboxylic acid moiety. The catheter of Example 1 was also much easier and less painful to insert and remove, in use. This major improvement is believed to have also been contributed to by the fact that the negatively charged carboxylate moiety is only able to act as a hydrogen bond acceptor and not a hydrogen bond donor. Conversely, the comparative additive with an —OH (alcohol) end-cap would be able to act as both a hydrogen bond donor and acceptor. Mucins in the outermost layer of the urinary bladder mucosa contain numerous hydrogen bond acceptor moieties, which would be able to form strong hydrogen bonds with an OH (alcohol) but not with a negatively charged carboxylate moiety, resulting in the inventive catheter displaying much lower “sticking effects” in use.

### Example 2

A second embodiment of an intermittent catheter of the invention is provided by an intermittent catheter comprising a hollow polymeric body comprising a base polymer formed of polyethylene and further comprising an A-B block copolymer amphiphilic additive of the formula CH₃CH₂(CH₂CH₂)₁₅(OCH₂CH₂)₅COOH present as a layer comprising the additive on the complete outer surface of the body of the catheter. The hydrophilic polyethylene oxide block of the additive similarly seeks away from the body and towards the external environment due to its incompatibility with the hydrophobic base polymer. The lipophilic hydrophobic polyethylene block of the additive ensures that the hydrophilic block is secured to the base material.

The intermittent catheter may be prepared as described in US patents U.S. Pat. No. 10,058,638 B2 and U.S. Pat. No. 9,186,438 B2, but with the added step of extrusion coating the layer comprising the additive on the outer surface of the catheter body after forming the body. This step can be performed using a blown or cast film process to coat the layer as a molten web of synthetic resin onto the outer surface of the intermittent catheter body after its formation. The process involves extruding the molten layer comprising the additive from a slot die directly onto a tacky catheter body that is moved beneath the die to form the layer on the outer surface of the catheter. The catheter is then cooled to bring the molten film of additive back into a solid/gel state and to completely solidify the tacky catheter body.

The layer of additive has a thickness of 100 μm and an additive concentration of 6% by weight of the combination of the base polymer and the layer of additive.

The catheter is contacted with an aqueous medium prior to use, as for Example 1 above, after which the catheter is used in the conventional manner.

Like Example 1, the intermittent catheter of Example 2 was less susceptible to bacterial attachment and was much easier and less painful to insert and remove, in use, compared with a catheter comprising a similar additive that was also present as a layer on the outer surface of the body, but which had a terminal —OH group instead of the carboxylic acid moiety.

The above embodiments are described by way of example only. Many variations are possible without departing from the scope of the invention as defined in the appended claims.

1. An A-B block copolymer amphiphilic additive comprising a hydrophobic hydrocarbon A-block and a hydrophilic poly (alkylene oxide) B-block, wherein the B-block is end-capped with a moiety that is negatively charged or that becomes negatively charged when wetted with an aqueous medium having a pH of greater than 2.

2. An A-B block copolymer amphiphilic additive as claimed in claim 1, wherein the additive comprises a poly (alkylene oxide) B-block formed from at least one alkylene oxide monomer unit that is independently selected from the group consisting of: ethylene oxide, propylene oxide, and combinations thereof.

3. An A-B block copolymer amphiphilic additive as claimed in claim 2, wherein the poly (alkylene oxide) is polyethylene oxide.

4. An A-B block copolymer amphiphilic additive as claimed in any preceding claim, wherein the poly (alkylene oxide) B-block contains between 2 and 10 alkylene oxide monomer units.

5. An A-B block copolymer amphiphilic additive as claimed in any preceding claim, wherein the A-block comprises a hydrocarbon chain block of the formula CH₃CH₂(CH₂CH₂)ₐ where “a” is 5-25 and preferably 9-25.

6. An A-B block copolymer amphiphilic additive as claimed in any preceding claim, wherein the B-block is end-capped with an ionisable moiety, preferably a deprotonatable moiety that becomes negatively charged when wetted with an aqueous medium having a pH of greater than 2.

7. An A-B block copolymer amphiphilic additive as claimed in any preceding claim, wherein the B-block is end-capped with a carboxylate-containing moiety.

8. An intermittent catheter comprising a hollow polymeric tubular body comprising a base polymer and a copolymer amphiphilic additive comprising at least one hydrophobic portion and at least one hydrophilic portion, wherein at least one hydrophilic portion comprises a moiety at a terminal end thereof that is negatively charged or that becomes negatively charged when wetted with an aqueous medium having a pH of greater than 2.

9. An intermittent catheter as claimed in claim 8, wherein the additive is a block copolymer comprising the at least one hydrophobic portion in the form of at least one hydrophobic A-block and further comprising the at least one hydrophilic portion in the form of at least one hydrophilic B-block, wherein at least one hydrophilic B-block comprises a moiety at a terminal end thereof that is negatively charged or that becomes negatively charged when wetted with an aqueous medium having a pH of greater than 2.

10. An intermittent catheter as claimed in claim 9, wherein the additive is an A-B block copolymer comprising a hydrophobic A-block and a hydrophilic B-block, said hydrophilic B-block comprising a moiety at its terminal end that is negatively charged or that becomes negatively charged when wetted with an aqueous medium having a pH of greater than 2.

11. An intermittent catheter as claimed in claim 9 or 10, wherein the additive is only negatively charged or only becomes negatively charged when wetted with an aqueous medium having a pH of greater than 2, at the terminal end of the or at least one B-block.

12. An intermittent catheter as claimed in claim 11, wherein less than or equal to 75% of the total number of B-block monomer units are negatively charged or become negatively charged when wetted with an aqueous medium having a pH of greater than 2, said monomer units being at the terminal end of the B-block.

13. An intermittent catheter as claimed in any one of claims 9 to 12, wherein the or at least one B-block is end-capped with a moiety that is negatively charged or that becomes negatively charged when wetted with an aqueous medium having a pH of greater than 2.

14. An intermittent catheter as claimed in claim 13, wherein only the end-cap moiety of the additive is negatively charged or becomes negatively charged when wetted with an aqueous medium having a pH of greater than 2.

15. An intermittent catheter as claimed in any one of claims 10 to 14, wherein the additive is the additive of any one of claims 1 to 7.

16. An intermittent catheter as claimed in any one of claims 8 to 15, wherein the base polymer is formed from at least one material that is independently selected from the group consisting of: polyolefins, polyesters, polyacrylates, polyamides, thermoplastic elastomeric material, polyether block amide, thermoplastic vulcanizates, thermoplastic copolyesters, thermoplastic polyamides, fluororubber, water disintegrable or enzymatically hydrolysable material, and combinations, blends or co-polymers of any of the above materials.

17. An intermittent catheter as claimed in claim 16, wherein the base polymer is formed from at least one polyolefin material, preferably comprising polyethylene and/or polypropylene.

18. An intermittent catheter as claimed in claim 16 or 17, wherein the base polymer is formed from at least one thermoplastic elastomeric material.

19. An intermittent catheter as claimed in any one of claims 8 to 18, wherein the additive is homogenously distributed with the polymer.

20. An intermittent catheter as claimed in any one of claims 8 to 19, wherein at least some of the additive is present at and/or on a surface of the catheter body, preferably an outer surface.

21. An intermittent catheter as claimed in claim 20, wherein at least 50% of the additive copolymers are present at and/or on the outer surface of the body.

22. An intermittent catheter as claimed in claim 20 or 21, wherein the moieties that are negatively charged or that become negatively charged when wetted with an aqueous medium having a pH of greater than 2, are present at and/or on at least 10% of the outer surface area of the body.

23. An intermittent catheter as claimed in any one of claims 8 to 22, wherein the additive comprises a layer that is on or comprises the outer surface of the body.

24. An intermittent catheter as claimed in any one of claims 8 to 23, wherein the additive is present at a concentration of at least 0.5% by weight of the combination of base polymer and additive.

25. An intermittent catheter as claimed in any one of claims 8 to 24, wherein the catheter comprises at least one further additive, and the copolymer additive comprising the moiety that is negatively charged or that becomes negatively charged when wetted with an aqueous medium having a pH of greater than 2 comprises at least 2% of the total number of additives.

26. Use of a copolymer amphiphilic additive comprising at least one hydrophobic portion and at least one hydrophilic portion, wherein at least one hydrophilic portion comprises a moiety at a terminal end thereof that is negatively charged or that becomes negatively charged when wetted with an aqueous medium having a pH of greater than 2, as a bacteria-repellent and/or lubricant in or on an intermittent catheter.

27. A method of manufacturing an intermittent catheter, the method comprising the steps of:
a. Providing a base polymer and a copolymer amphiphilic additive comprising at least one hydrophobic portion and at least one hydrophilic portion, wherein at least one hydrophilic portion comprises a moiety at a terminal end thereof that is negatively charged or that becomes negatively charged when wetted with an aqueous medium having a pH of greater than 2; and
b. Forming a hollow polymeric tubular catheter body comprising the base polymer and the additive.
